(19) United States
(12) Patent Application Publication
(10) Pub. No.: US 2016/0023011 A1
(43) Pub. Date: Jan. 28, 2016

(54) METHODS AND SYSTEMS FOR CARDIAC STIMULATION

(71) Applicant: PACESETTER, INC., Sylmar, CA (US)

(72) Inventors: Gene A. Bornzin, Simi Valley, CA (US); David E. Anderson, Castaic, CA (US)

(21) Appl. No.: 14/340,025

(22) Filed: Jul. 24, 2014

(51) Int. Cl. A61N 1/39; A61N 1/365

(52) U.S. Cl. CPC A61N 1/3962; A61N 1/3987; A61N 1/36592

(57) ABSTRACT

The present disclosure provides systems and methods for cardiac stimulation. The cardiac stimulation system includes a first cardiac stimulation device implanted in a subject, and a second cardiac stimulation device implanted in the subject and communicatively coupled to the first cardiac stimulation device. The cardiac stimulation system is configured to detect an arrhythmia in the subject, and apply a shocking pulse in response to the detected arrhythmia by supplying a first output voltage from the first cardiac stimulation device and a second output voltage from the second cardiac stimulation device, wherein the first output voltage has a first polarity and the second output voltage has a second polarity that is opposite from the first polarity.

### A. FIELD OF THE DISCLOSURE

The present disclosure relates generally to cardiac stimulation systems, and more particularly to cardiac stimulation systems including two separate stimulation devices.

### B. BACKGROUND ART

Defibrillators deliver pulses of electrical energy to a subject's heart to treat life-threatening cardiac arrhythmia and other cardiac phenomena. The electrical energy terminates the arrhythmia and allows a normal sinus rhythm to be reestablished. Defibrillators may be external or implanted devices.

FIG. 1 illustrates a known implantable medical system 9 capable of delivering pulses for defibrillation and/or pacing. In this example, implantable medical system 9 includes a pacer/ICD 10 or other cardiac stimulation device (such as a CRT device) equipped with a set of cardiac sensing/pacing leads 12 implanted on or within the heart of the patient, including at least an RV lead and an LV lead implanted via the coronary sinus (CS) for biventricular pacing. In FIG. 1, a stylized representation of the leads is set forth. In the case where pacer/ICD 10 is intended to operate as an implantable cardioverter/defibrillator (ICD) device, it detects the occurrence of an arrhythmia in the patient, and automatically applies an appropriate electrical shock therapy to the heart aimed at terminating the detected arrhythmia.

Pacer/ICD 10 is programmed using an external programming device 14 under clinician control and/or patient control. External programming device 14 may be a bedside monitor or other diagnostic device such as a personal advisory module (PAM) that receives and displays data from pacer/ICD 10. Bedside monitor 14 may be directly networked with a centralized computing system, such as the HouseCall™ system or the Merlin@home/Merlin.Net systems of St. Jude Medical, which can relay diagnostic information to the clinician.

The size of pacer/ICD 10 may be limited by the ability of pacer/ICD 10 to deliver electrical pulses to a subject. For example, in order to apply relatively strong electrical pulses, at least some known implantable defibrillators include at least four capacitors for storing and discharging the electrical energy. The space occupied by these capacitors causes such implantable defibrillators to be relatively large, increases complexity of the implantation and may cause discomfort to the subject. Accordingly, it would be desirable to have an implantable cardiac stimulation system that is able to provide substantially the same magnitude of electrical pulses using relatively smaller components.

### BRIEF SUMMARY OF THE DISCLOSURE

In one embodiment, the present disclosure is directed to a cardiac stimulation system. The cardiac stimulation system includes a first cardiac stimulation device implanted in a subject, and a second cardiac stimulation device implanted in the subject and communicatively coupled to the first cardiac stimulation device. The cardiac stimulation system is configured to detect an arrhythmia in the subject, and apply a shocking pulse in response to the detected arrhythmia by supplying a first output voltage from the first cardiac stimulation device and a second output voltage from the second cardiac stimulation device, wherein the first output voltage has a first polarity and the second output voltage has a second polarity that is opposite from the first polarity.

In another embodiment, the present disclosure is directed to a method for applying cardiac stimulation to a subject. The method includes detecting an arrhythmia using a first cardiac stimulation device implanted in the subject, supplying, from the first cardiac stimulation device, a first output voltage at a first polarity, and supplying, from a second cardiac stimulation device implanted in the subject and communicatively coupled to the first cardiac stimulation device, a second output voltage at a second polarity, wherein the second polarity is opposite from the first polarity, wherein the first and second output voltages form a shocking pulse.

In another embodiment, the present disclosure is directed to a cardiac stimulation system. The cardiac stimulation system includes a first cardiac stimulation device configured to be implanted in a subject, a second cardiac stimulation device configured to be implanted in the subject, and a subcutaneous connection wire communicatively coupling the first cardiac stimulation device to the second cardiac stimulation device, wherein said first cardiac stimulation device is configured to supply a first output voltage to the subject and the second stimulation device is configured to supply a second output voltage to the subject, the first and second output voltages forming a shocking pulse configured to disrupt an arrhythmia.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic diagram of a known implantable medical system.

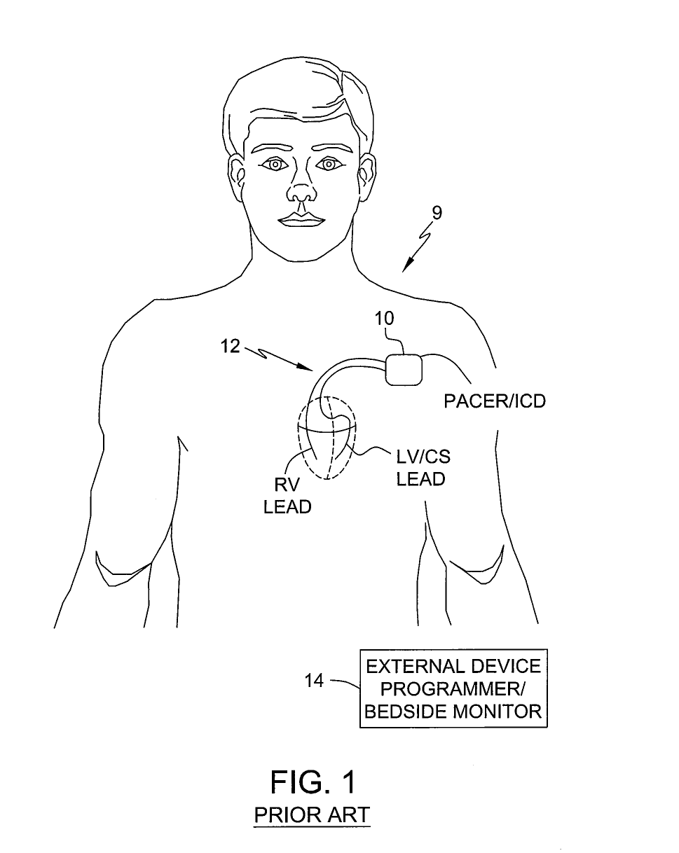

FIG. 2 is a schematic diagram of a cardiac stimulation system.

FIG. 3 is a schematic diagram of the cardiac stimulation system shown in FIG. 1 implanted in a patient.

FIGS. 4A and 4B are voltage traces for a shocking pulse delivered using the cardiac stimulation system shown in FIG. 1.

FIG. 5 is a schematic circuit diagram of the cardiac stimulation system shown in FIG. 1.

FIG. 6 is a schematic circuit diagram of an alternative cardiac stimulation system.

FIG. 7 is a flow diagram for applying cardiac stimulation to a patient.

Corresponding reference characters indicate corresponding parts throughout the several views of the drawings.

### DETAILED DESCRIPTION OF THE DISCLOSURE

The present disclosure provides systems and methods for applying cardiac stimulation to a subject. The cardiac stimulation system includes a first cardiac stimulation device implanted in a subject, and a second cardiac stimulation device implanted in the subject and communicatively coupled to the first cardiac stimulation device. The cardiac stimulation system is configured to detect an arrhythmia in the subject, and apply a shocking pulse in response to the detected arrhythmia by supplying a first output voltage from the first cardiac stimulation device and a second output voltage from the second cardiac stimulation device, wherein the first output voltage has a first polarity and the second output voltage has a second polarity that is opposite from the first polarity.

Unless defined otherwise, technical and scientific terms used herein have the same meaning as commonly understood by one of ordinary skill in the art to which this disclosure belongs. For purposes of the present disclosure, the following terms are defined below.

As used herein, the use of the word “a” or “an” when used in conjunction with the term “comprising” in the claims and/or the specification may mean “one”, but it is also consistent with the meaning of “one or more,” “at least one”, and “one or more than one”. Still further, the terms “having”, “including”, “containing” and “comprising” are interchangeable and one of skill in the art is cognizant that these terms are open-ended terms. Some embodiments may consist of or consist essentially of one or more elements, method steps, and/or methods of the disclosure. It is contemplated that any method or composition described herein can be implemented with respect to any other method or composition described herein.

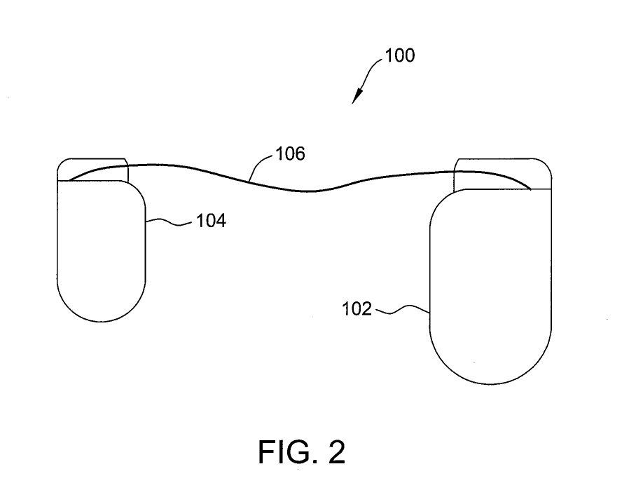

FIG. 2 is a schematic diagram of a cardiac stimulation system 100 according to one embodiment. Cardiac stimulation system 100 includes a first stimulation device 102 and a second stimulation device 104. In this embodiment, first stimulation device 102 is an implantable cardioverter/defibrillator (ICD), and second cardiac stimulation device 104 is a driver device, also referred to as a captrode (i.e., a combined capacitor and electrode), as described in detail herein. Alternatively, first and second stimulation devices 102 and 104 may be any devices that enable system 100 to function as described herein. In the embodiment shown in FIG. 2, first and second stimulation devices 102 and 104 are coupled to one another using at least one cable 106 that facilitates communication between first and second stimulation devices 102 and 104. Alternatively, first and second stimulation devices 102 and 104 may communicate with one another using other suitable communication techniques.

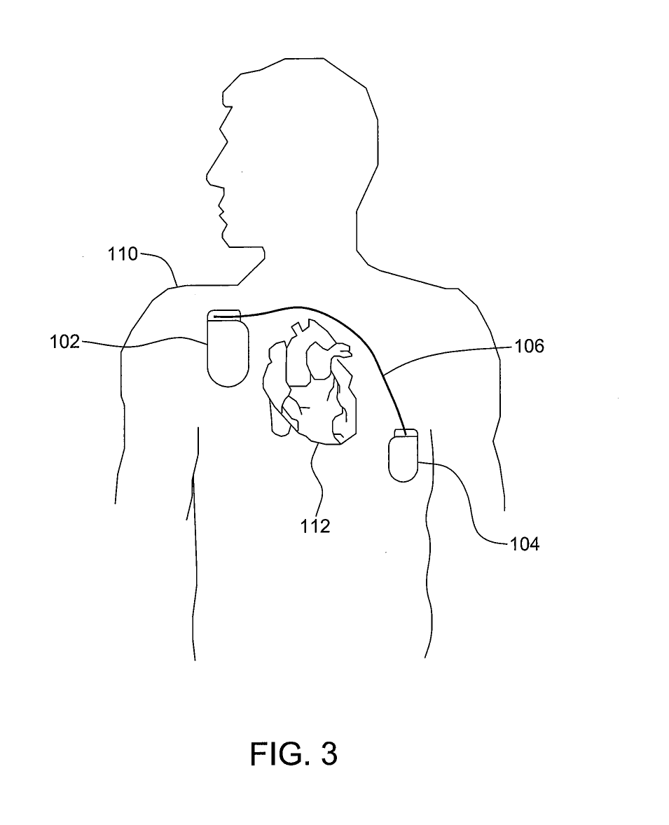

To apply cardiac stimulation, first and second stimulation devices 102 and 104 are implanted within a patient 110, as shown in FIG. 3. First and second stimulation devices 102 and 104 may be implanted at positions similar to positions where ICD paddles would typically be placed for defibrillation. For example, in the embodiment shown in FIG. 3, first stimulation device 102 is positioned on a right side of patient 110 and above the heart 112, and second stimulation device 104 is positioned on a left side of patient 110 and below heart 112. Accordingly, heart 112 is positioned between first and second stimulation devices 102 and 104 when a voltage difference is applied between first and second stimulation devices 102, as described herein. Alternatively, first and second stimulation devices 102 and 104 may be located at any positions that enable system 100 to function as described herein.

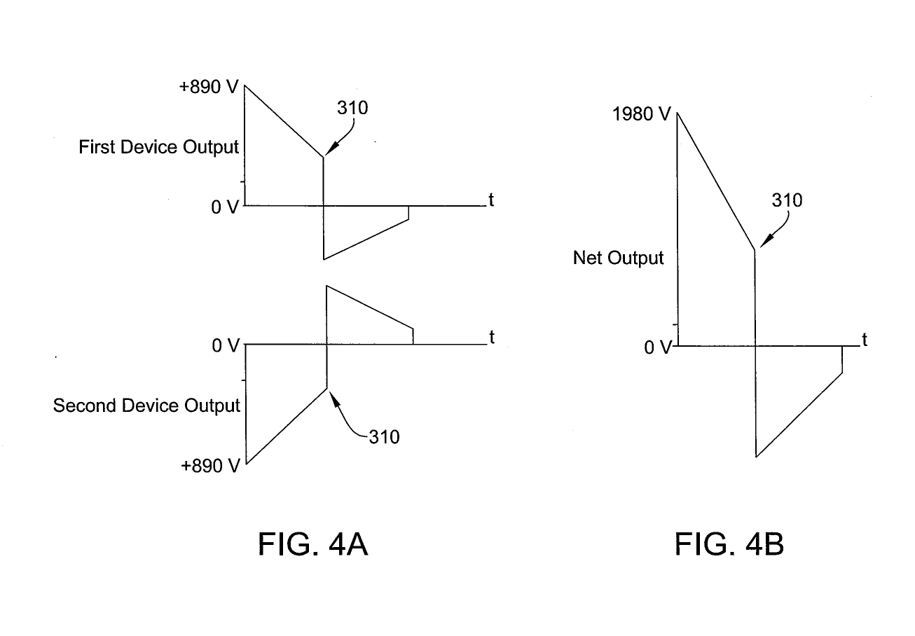

FIGS. 4A and 4B are voltage traces for a shocking (i.e., defibrillating) pulse according to one embodiment. More specifically, FIG. 4A illustrates separate voltage outputs from first stimulation device 102 and second stimulation device 104, and FIG. 4B illustrates the total output when the traces in FIG. 4A are combined. The shocking pulse may deliver, for example, eighty joules of energy to patient 110, with each of first and second stimulation devices 102 and 104 supplying forty joules.

In this embodiment, to produce the shocking pulse, first stimulation device 102 generates an initial output voltage at a first polarity (e.g., +890 Volts (V)), and second stimulation device 104 generates an initial output voltage at a second polarity opposite the first polarity (e.g., −890 V). By generating output voltages having opposite polarity, the output voltage of first stimulation device 102 and the output voltage of second stimulation device 104 combine to a larger total initial output voltage (e.g., 1980 V) than either device may be capable of individually.

In this embodiment, although the polarities of the output voltages are opposite, the magnitude of the two output voltages is the same (i.e., 890 V). Alternatively, the output voltage of first stimulation device 102 may be different (i.e., greater than or less than) the output voltage of second stimulation device 104. Further, first stimulation device 102 and second stimulation device 104 may have any output voltages that enable system 100 to function as described herein.

As shown in FIGS. 4A and 4B, the output voltages of first and second stimulation devices 102 and 104 decay linearly over time. At a trigger point 310, the polarity of the output voltages for first and second stimulation devices 102 and 104 switch. That is, if the output voltage for first stimulation device 102 is initially positive and the output voltage for second stimulation device 104 is initially negative, at trigger point 310, the output voltage for first stimulation device 102 becomes negative, and the output voltage for second stimulation device 104 becomes positive. Because of the polarity switch, the voltage output of system 100 may be referred to as a push pull output. As used herein, the portion of the pulse before trigger point 310 is referred to the first phase of the shocking pulse, and the portion of the pulse after trigger point 310 is referred to as the second phase of the shocking pulse.

In this embodiment, trigger point 310 is set at a point where the output voltage has dropped 65% (i.e., a 65%//35% fixed tilt). Alternatively, trigger point 310 may be set at any point that enables system 100 to function as described herein. For example, in some embodiments, trigger point 310 is set using a 50%/50% fixed tilt. Further, in some embodiments, the setting for trigger point 310 is patient specific.

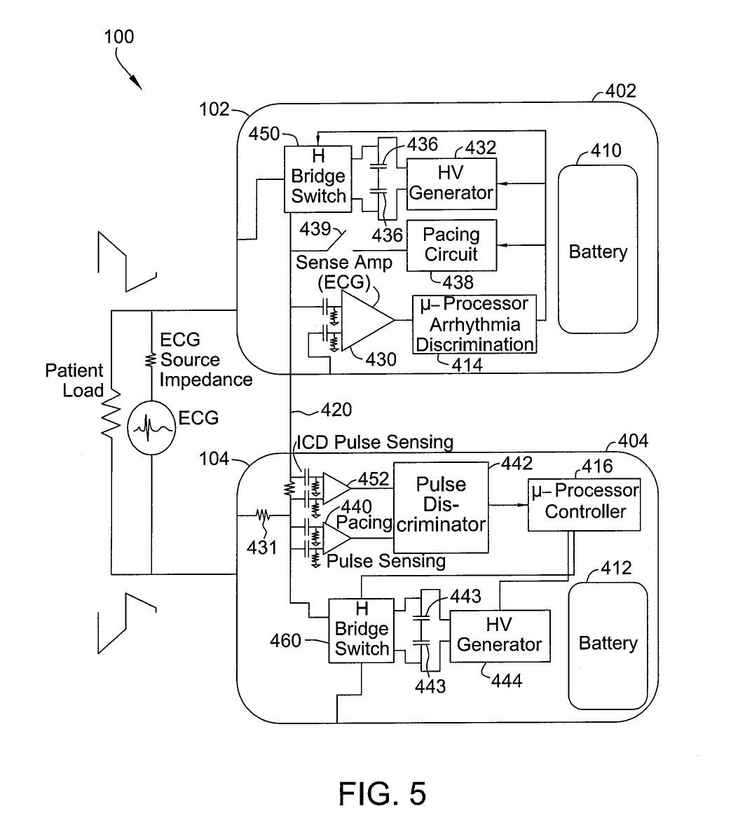

FIG. 5 is a schematic circuit diagram of system 100. As shown in FIG. 5, first stimulation device 102 includes a first housing 402 and second stimulation device 104 includes a second housing 404. In some embodiments, each of first and second housings 402 and 404 is a titanium case. Alternatively, first and second housing 402 and 404 may be any enclosures that enable system 100 to function as described herein. As noted above, in this embodiment, first stimulation device 102 is an ICD and second stimulation device 104 is a driver device

First stimulation device 102 includes a battery 410 that provides power to one or more components in first stimulation device 102. Similarly, in the embodiment shown in FIG. 5, second stimulation also includes a battery 412. The type of batteries 410 and 412 may vary depending on the capabilities of first and second stimulation devices 102 and 104. For example, to employ shocking therapy, the batteries 410 and 412 may be capable of operating at low current drains for long periods, and also capable of providing high-current pulses (for capacitor charging) when patient 110 requires a shock pulse. Batteries 410 and 412 may also have a predictable discharge characteristic so that an elective replacement time can be detected. Accordingly, appropriate batteries 410 and 412 are employed in system 100. In some embodiments, battery 412 is smaller than battery 410, because second stimulation device 104, unlike first stimulation device 102, does not perform arrhythmia discrimination.

First and second stimulation devices 102 and 104 each include associated processing devices 414 and 416 in this embodiment. Processing devices 414 and 416 control operation of first and second stimulation devices 102 and 104, respectively. Processing devices 414 and 416 include a microprocessor, or equivalent control circuitry, designed specifically for controlling the delivery of stimulation therapy and may further include RAM or ROM memory, logic and timing circuitry, state machine circuitry, and/or I/O circuitry. In this embodiment, processing devices 414 and 416 are capable of processing and monitoring input signals (data) as controlled by a program code stored in a designated block of memory. Any suitable processing devices 414 and 416 may be used that carry out the functions described herein.

First and second stimulation devices 102 and 104 are communicatively coupled to one another. To facilitate this, in this embodiment, first and second stimulation devices 102 and 104 are coupled to each other via a subcutaneous connection wire 420. Alternatively, first and second stimulation devices 102 may be communicatively coupled to one another using any suitable wired and/or wireless communication methods.

In the embodiment shown in FIG. 5 first stimulation device 102 detects an arrhythmia in patient 110 using a sense amp 430 and processing device 414. Sense amp 430 records an echocardiogram (ECG) by monitoring electrical activity across the load created by patient 110 (typically 50 to 80 ohms). Specifically, in this embodiment, sense amp 430 is electrically coupled to first housing 402 and, through a resistor 431, to second housing 404. Resistor 431 may be, for example, a 500 ohm resistor. Signals from sense amp 430 are digitized and processed with an automatic gain control (AGC) system (now shown), and supplied to processing device 414. Based on the signals, processing device 414 determines whether a shockable arrhythmia occurs in patient 110.

When processing device 414 detects a shockable arrhythmia, a high-voltage (HV) generator 432 charges capacitors 436 to prepare first stimulation device 102 for applying the shocking pulse to patient 110. Upon detection of the shockable arrhythmia, in this embodiment, processing device 414 also causes first stimulation device 102 to send a pacing pulse to second stimulation device 104. Alternatively, first stimulation device 102 may communicate with second stimulation device 104 other than using pacing pulses. For example, in some embodiments, first and second stimulation devices 102 and 104 may exchange complex serial messages.

The pacing pulse may be, for example, a small level negative pulse generated by a pacing circuit 438 and transmitted through subcutaneous connection wire 420 when a switch 439 is closed. A pacing pulse sensing circuit 440 in second stimulation device 104 detects the pacing pulse, and a pulse discriminator 442 notifies processing device 416. In response, processing device 416 causes a HV generator 444 to charge capacitors 443 on second stimulation device 104.

Once capacitors 436 and 443 are charged, first stimulation device 102 initiates delivery of the first phase of the shocking pulse. Specifically, capacitors 436 begin discharging, with an H bridge switch 450 controlling the polarity of the discharged voltage. Using an ICD pulse sensing circuit 452, second stimulation device 104 detects the initiation of the first phase delivered from first stimulation device 102, and pulse discriminator 442 notifies processing device 416. In response, second stimulation device 104 begins delivery of its opposite polarity pulse by discharging capacitors 443. As in first stimulation device 102, an H bridge switch 460 controls the polarity of the discharged voltage.

Thus, second stimulation device 104 discharges capacitors 443 after it detects discharging of capacitors 436. In this embodiment, capacitors 443 begin discharging microseconds after capacitors 436 begin discharging. Accordingly, first and second stimulation devices 104 initiate their opposite polarity outputs near-instantaneously.

At the trigger point 310 (shown in FIGS. 4A and 4B), the polarity of the output voltage of first stimulation device 102 switches (e.g., using H bridge switch 450), initiating the second phase of the shocking pulse. Similar to the first phase, second stimulation device 104 detects (i.e., using ICD pulse sensing circuit 452) the initiation of the second phase by first stimulation device 104. In response, the polarity of the output voltage of second stimulation device 104 switches (e.g., using H bridge switch 460), initiating the second phase of the shocking pulse for second stimulation device 104. As with the first phase, first and second stimulation devices 102 and 104 initiate their opposite polarity voltage outputs near-instantaneously for the second phase.

At a predetermined point (e.g., when capacitors 436 are substantially discharged), first stimulation device 102 finishes the second phase, and stops supplying an output voltage. Second stimulation device 104 detects (i.e., using ICD pulse sensing circuit 452) that the second phase has ended for first stimulation device 102. In response, second stimulation device 104 also ends the second phase. As with the first and second phases, first and second stimulation devices 102 and 104 end the second phase near-instantaneously.

After first and second stimulation devices 102 and 104 are finished delivering the shocking pulse, first stimulation device 102 resumes monitoring patient 110 to detect shockable arrhythmias. In some embodiments, at least one of first and second stimulation devices 102 and 104 applies post-shock pacing to patient 110. For example, if the inter-beat (RR) interval is greater than one second, first stimulation device 102 may deliver post-shock pacing for up to thirty seconds.

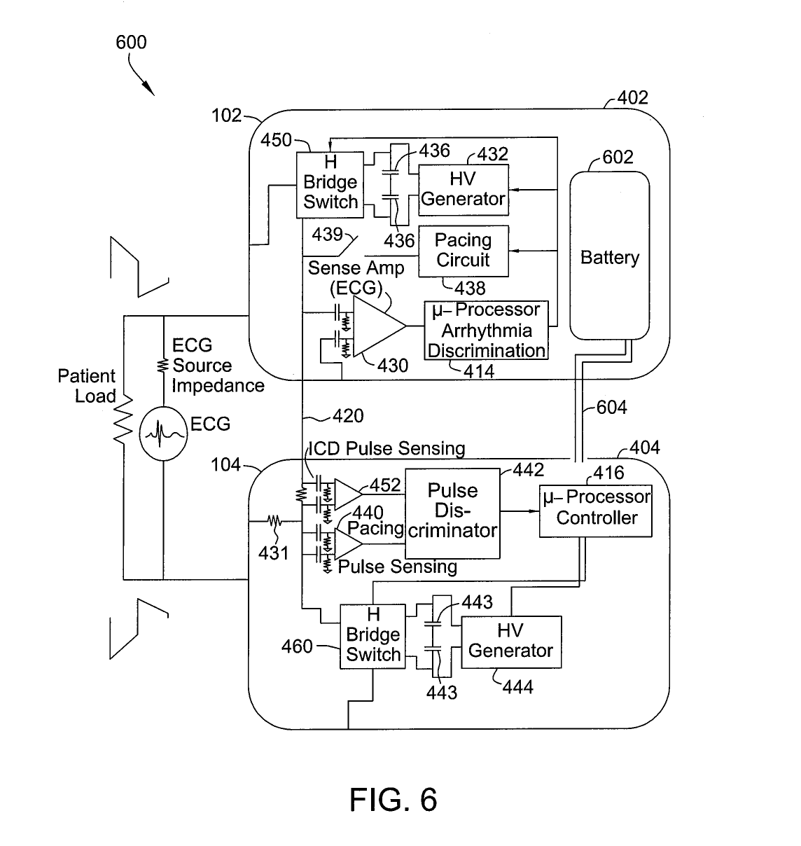

FIG. 6 is an alternative embodiment 600 of system 100. Unless otherwise indicated, system 600 operates substantially similar to system 100. Notably, in system 600, second stimulation device 104 does not include an associated battery. Instead, a shared battery 602 on first stimulation device 102 provides power to components of both first and second stimulation devices 102 and 104. Specifically, second stimulation device 104 receives power from a power cable 604 coupled between shared battery 602 and second stimulation device 104. Although the embodiment shown in FIG. 6 adds additional electrical connections (i.e., power cable 604) it simplifies second stimulation device 104 and reduces the number of batteries required.

Notably, the embodiments of first and second stimulation devices 102 and 104 shown in FIGS. 5 and 6 are merely exemplary. Those of skill in the art will appreciate that modifications (e.g., using different components and/or configurations) may be made to first and second stimulation devices 102 and 104 without departing form the spirit and scope of the disclosure.

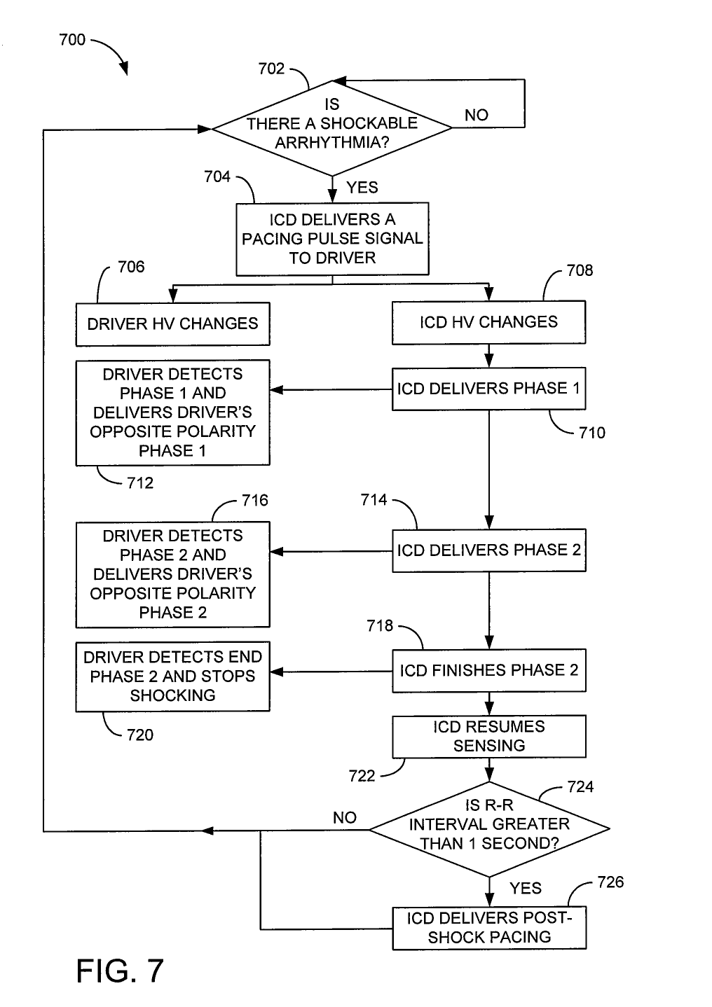

FIG. 7 is a flow diagram 700 for applying cardiac stimulation to a patient. Flow diagram 700 may be implemented, for example, using system 100 and/or system 600. At block 702, a first stimulation device (e.g., an ICD) determines whether there is a shockable arrhythmia. If there is no shockable arrhythmia, flow returns to block 702, and the ICD continues to monitor for shockable arrhythmias.

If there is a shockable arrhythmia, flow proceeds to block 704, and the ICD delivers a pacing pulse signal to a second stimulation device (e.g., a driver). Further, the driver charges (i.e., by charging capacitors) at block 706, and the ICD charges at block 708. At block 710, the ICD initiates delivery of the first phase at a first polarity. At block 712, the driver detects the initiation by the ICD and initiates its own portion of the first phase at a second polarity opposite the first polarity.

At block 714, the ICD initiates delivery of the second phase, switching the output voltage from the first polarity to the second polarity. At block 716, the driver detects the switch to the second phase, and switches its own output voltage from the second polarity to the first polarity. At block 718, the ICD finishes the second phase. At block 720, the driver detects that the ICD has finished the second phase, and stops providing an output voltage, ending the shocking pulse.

Flow then proceeds to block 722, in which the ICD resumes monitoring the patient for shockable arrhythmia. At block 724, if the RR interval is not greater than one second, flow returns to block 702. If the RR interval is greater than one second, flow proceeds to block 726, where the ICD delivers post-shock pacing. Alternatively, in some embodiments, the driver may deliver the post-shock pacing. Once the post-shock pacing is completed (e.g., after thirty seconds) flow returns to block 702.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A cardiac stimulation system comprising:
a first cardiac stimulation device implanted in a subject; and
a second cardiac stimulation device implanted in the subject and communicatively coupled to the first cardiac stimulation device, wherein the cardiac stimulation system is configured to:
detect an arrhythmia in the subject; and
apply a shocking pulse in response to the detected arrhythmia by supplying a first output voltage from the first cardiac stimulation device and a second output voltage from the second cardiac stimulation device, wherein the first output voltage has a first polarity and the second output voltage has a second polarity that is opposite from the first polarity.

2. The system of claim 1, wherein the first cardiac simulation device comprises a battery configured to provide power to both the first cardiac stimulation device and the second cardiac stimulation device.

3. The system of claim 1, wherein a magnitude of the first output voltage is approximately equal to a magnitude of the second output voltage.

4. The system of claim 1, wherein the first cardiac stimulation device is configured to:
switch the polarity of the first output voltage from the first polarity to the second polarity when the first output voltage reaches a predetermined voltage level, and wherein the second cardiac stimulation device is configured to detect the switch in polarity of the first output voltage, and switch the polarity of the second output voltage from the second polarity to the first polarity in response to the detection.

5. The system of claim 1, wherein the first cardiac stimulation device is an ICD.

6. The system of claim 1, further comprising a subcutaneous connection wire communicatively coupling the first and second cardiac stimulation devices.

7. The system of claim 1, wherein the second cardiac stimulation device is configured to:
detect that the first cardiac stimulation device is supplying the first output voltage; and
supply the second output voltage in response to the detection.

8. A method for applying cardiac stimulation to a subject, the method comprising:
detecting an arrhythmia using a first cardiac stimulation device implanted in the subject;
supplying, from the first cardiac stimulation device, a first output voltage at a first polarity; and
supplying, from a second cardiac stimulation device implanted in the subject and communicatively coupled to the first cardiac stimulation device, a second output voltage at a second polarity, wherein the second polarity is opposite from the first polarity, wherein the first and second output voltages form a shocking pulse.

9. The method of claim 8, further comprising providing power to both the first and second cardiac stimulation devices using a battery located within the first cardiac stimulation device.

10. The method of claim 8, wherein a magnitude of the first output voltage is approximately equal to a magnitude of the second output voltage.

11. The method of claim 8, further comprising:
switching the polarity of the first output voltage from the first polarity to the second polarity when the first output voltage reaches a predetermined voltage level;
detecting, at the second cardiac stimulation device, the switch in polarity of the first output voltage; and
switching the polarity of the second output voltage from the second polarity to the first polarity in response to the detection.

12. The method of claim 8, wherein supplying a first output voltage comprises supplying the first output voltage from an ICD.

13. The method of claim 8, further comprising coupling a subcutaneous connection wire between the first and second cardiac stimulation devices to communicatively couple the first and second cardiac stimulation devices.

14. The method of claim 8, wherein supplying a second output voltage comprises:
detecting, at the second cardiac stimulation device, that the first cardiac stimulation device is supplying the first output voltage; and
supplying the second output voltage in response to the detection.

15. The method of claim 8, further comprising:
charging the first cardiac stimulation device in response to the arrhythmia detection;
transmitting a pacing pulse from the first cardiac stimulation device to the second cardiac stimulation device in response to the arrhythmia detection; and
charging the second cardiac stimulation device in response to receiving the pacing pulse.

16. The method of claim 8, further comprising applying post-shock pacing to the subject when, after application of the shocking pulse, a RR interval is greater than a predetermined time period.

17. The method of claim 8, where applying post-shock pacing comprises applying post-shock pacing using at least one of the first and second cardiac stimulation devices.

18. A cardiac stimulation system comprising:
a first cardiac stimulation device configured to be implanted in a subject;
a second cardiac stimulation device configured to be implanted in the subject; and
a subcutaneous connection wire communicatively coupling the first cardiac stimulation device to the second cardiac stimulation device, wherein said first cardiac stimulation device is configured to supply a first output voltage to the subject and the second stimulation device is configured to supply a second output voltage to the subject, the first and second output voltages forming a shocking pulse configured to disrupt an arrhythmia.

19. The system of claim 18, further comprising a battery configured to provide power to both the first and second cardiac stimulation devices.

20. The system of claim 18, wherein the first cardiac stimulation device is configured to communicate with the second communication device by transmitting pacing pulses through the subcutaneous connection wire.
